(19) United States
(12) Patent Application Publication
(10) Pub. No.: US 2023/0256200 A1
(43) Pub. Date: Aug. 17, 2023

(54) Vascular Access Blood Draw Device with Integrated Point-of-Care Small Volume Blood Collection Device

(71) Applicant: Becton, Dickinson and Company, Franklin Lakes, NJ (US)

(72) Inventors: John M. Lackey, West Valley City, UT (US); Megan S. Scherich, Salt Lake City, UT (US); Jonathan Karl Burkholz, Salt Lake City, UT (US); Erica E. Neumann, Sandy, UT (US)

(21) Appl. No.: 18/108,980

(22) Filed: Feb. 13, 2023

Related U.S. Application Data
(60) Provisional application No. 63/309,904, filed on Feb. 14, 2022.

(51) Int. Cl. A61M 25/01; A61M 39/10; A61M 39/22; A61B 5/15

(52) U.S. Cl. CPC A61M 25/0102; A61M 39/10; A61M 39/22; A61B 5/150099; A61B 5/150343; A61B 5/150992

(57) ABSTRACT

An instrument advancement device may include a housing comprising a distal end and a proximal end; an instrument disposed within the housing; an advancement element, wherein in response to movement of the advancement element with respect to the housing, the instrument is configured to advance distal to the distal end of the housing, and a blood sample collection tube inserted into the distal end of the housing, wherein the blood sample collection tube is configured to receive a small volume blood sample.

### CROSS-REFERENCE TO RELATED APPLICATION

The present application claims priority to U.S. Provisional Application No. 63/309,904, entitled “Vascular Access Blood Draw Device with Integrated Point-Of-Care Small Volume Blood Collection Device” filed Feb. 14, 2022, the entire disclosure of which is hereby incorporate by reference in its entirety.

### BACKGROUND OF THE INVENTION

### Field of the Invention

The present disclosure is directed to a vascular access blood draw device and, in particular, a vascular access blood draw device with an integrated point-of-care small volume blood collection device.

### Description of Related Art

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter device includes a catheter that is over-the-needle. As its name implies, the catheter that is over-the-needle may be mounted over an introducer needle having a sharp distal tip. A catheter assembly may include a catheter adapter, the catheter extending distally from the catheter adapter, and the introducer needle extending through the catheter. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

Recent developments in the peripheral intravenous catheter (PIVC) field have led to the emergence of technologies designed to facilitate blood draw and an in-dwelling PIVC. These devices have focused on the ability to reliably collect a high-quality blood sample and reduce hemolysis. The main method by which these devices work is by inserting a guide wire, probe, tube, or other instrument through the lumen of the catheter. This arrangement creates a fluid path through any thrombus or fibrin that may be occluding the tip of the catheter. A syringe or vacutainer may then be used to collect blood samples without needing to subject the patient to additional needle sticks.

Point-of-care (POC) testing includes the use of a portable testing system that can quickly deliver results using small volume blood samples, such as for glucose testing. Blood samples are typically collected via finger pricks or through existing vascular access. However, the majority of vascular access devices are not optimized for collection of small blood samples. The current field is moving toward miniaturized and personalized healthcare with the goal of POC testing that can provide rapid diagnostic results with a single drop of blood. There is a current need to provide POC blood sample collection options from a PIVC in anticipation for increased POC diagnostic testing.

Infusion and blood withdrawal using the catheter may be difficult for several reasons, particularly when an indwelling time of the catheter increases. A fibrin sheath or thrombus may form on an internal surface of the catheter assembly, an external surface of the catheter assembly, or within the vasculature near the distal tip of the catheter. The fibrin sheath or thrombus may block or narrow a fluid pathway through the catheter, which may impair infusion and/or collection of a high-quality blood sample.

The subject matter claimed herein is not limited to embodiments that solve any disadvantages or that operate only in environments such as those described above. Rather, this background is only provided to illustrate one example technology area where some implementations described herein may be practiced.

### SUMMARY OF THE INVENTION

In one non-limiting embodiment or aspect of the present disclosure, an instrument advancement device, comprising a housing comprising a distal end and a proximal end; an instrument disposed within the housing; an advancement element, wherein in response to movement of the advancement element with respect to the housing, the instrument is configured to advance distal to the distal end of the housing, and a blood sample collection tube inserted into the distal end of the housing, wherein the blood sample collection tube is configured to receive a small volume blood sample of 0.3 to 500 microliters.

In some non-limiting embodiments or aspects of the present disclosure, the small volume blood sample is 0.3 to 100 microliters. The instrument may include a septum providing in a proximal end of the instrument, and wherein the blood sample collection tube is inserted into the instrument through the septum to receive the small volume blood sample. After the small volume blood sample has been received in the blood sample collection tube, the blood sample collection tube is configured to be removed from the housing. The blood sample collection tube is configured to be operatively connected to a point-of-care system to test the small volume blood sample. The blood sample collection tube includes a flexible member on a proximal end of the blood sample collection tube to create suction to pull the small volume blood sample into the blood sample collection tube.

In one non-limiting embodiment or aspect of the present disclosure, an instrument advancement device, comprising a housing comprising a distal end and a proximal end; an instrument disposed within the housing; an advancement element, wherein in response to movement of the advancement element with respect to the housing, the instrument is configured to advance distal to the distal end of the housing, and a blood sample collection device integrated into the distal end of the housing.

In some non-limiting embodiments or aspects of the present disclosure, an opening of the blood sample collection device is in fluid communication with a fluid path defined by the instrument. The blood sample collection device is configured for removal from the housing once a small volume blood sample has been received in the blood sample collection device for point-of-care testing. The small volume blood sample may be 0.3 to 500 microliters or 0.3 to 100 microliters. A fluid chamber access port is defined in the distal end of the housing to receive the blood sample collection device. The fluid chamber access port includes a seal that prevents blood from exiting the housing after the blood sample collection device has been removed from the housing. The blood sample collection device is pre-attached to the fluid chamber access port. The fluid chamber access port includes a valve member that moves between an open position in which blood is free to flow into the blood sample collection device and a closed position in which the blood is prevented from entering the blood sample collection device.

In one non-limiting embodiment or aspect, an instrument advancement device, comprising a housing comprising a distal end and a proximal end; an instrument disposed within the housing; an advancement element, wherein in response to movement of the advancement element with respect to the housing, the instrument is configured to advance distal to the distal end of the housing, a blood sample access port defined in the housing, and an inner cylinder positioned within the housing that is rotatable relative to the housing.

In some non-limiting embodiments or aspects of the present disclosure, the inner cylinder defines an aperture, in which the aperture is rotatable into alignment with the blood sample access port to permit blood to flow into a blood sample collection device operatively connected to the instrument advancement device and out of alignment with the blood sample access port to prevent the blood flowing into the blood sample collection device. The housing further comprises a handle operatively connected to the inner cylinder to rotate the inner cylinder relative to the housing. The inner cylinder defines an aperture, and wherein blood sample access port is rotatable into alignment with the aperture to permit blood to flow into a blood sample collection device operatively connected to the instrument advancement device and out of alignment with the aperture to prevent the blood flowing into the blood sample collection device.

In one non-limiting embodiment or aspect of the present disclosure, an instrument advancement device, comprising a housing comprising a distal end and a proximal end; an instrument disposed within the housing; an advancement element, wherein in response to movement of the advancement element with respect to the housing, the instrument is configured to advance distal to the distal end of the housing, and a blood sample collection device operatively connected to the advancement element to collect a small volume blood sample of 0.3 to 500 microliters.

In some non-limiting embodiments or aspects of the present disclosure, the blood sample collection device is operatively connected to a proximal end of the advancement element. The advancement element defines a fluid path to fluidly connect the blood sample collection device to a fluid path defined by the instrument. The blood sample collection device is configured to be removed from the advancement element after the small volume blood sample has been received in the blood sample collection device for analysis by a point-of-care device. The small volume blood sample may be 0.3 to 100 microliters.

The following numbered clauses are also directed to the present disclosure:

Clause 1: An instrument advancement device, comprising a housing comprising a distal end and a proximal end; an instrument disposed within the housing; an advancement element, wherein in response to movement of the advancement element with respect to the housing, the instrument is configured to advance distal to the distal end of the housing, and a blood sample collection tube inserted into the distal end of the housing, wherein the blood sample collection tube is configured to receive a small volume blood sample of 0.3 to 500 microliters or 0.3 to 100 microliters.

Clause 2: The instrument advancement device of Clause 1, wherein the instrument includes a septum providing in a proximal end of the instrument, and wherein the blood sample collection tube is inserted into the instrument through the septum to receive the small volume blood sample.

Clause 3: The instrument advancement device of Clause 1 or Clause 2, wherein, after the small volume blood sample has been received in the blood sample collection tube, the blood sample collection tube is configured to be removed from the housing.

Clause 4: The instrument advancement device of Clause 3, wherein the blood sample collection tube is configured to be operatively connected to a point-of-care system to test the small volume blood sample.

Clause 5: The instrument advancement device of any of Clauses 1-4, wherein the blood sample collection tube includes a flexible member on a proximal end of the blood sample collection tube to create suction to pull the small volume blood sample into the blood sample collection tube.

Clause 6: An instrument advancement device, comprising a housing comprising a distal end and a proximal end; an instrument disposed within the housing; an advancement element, wherein in response to movement of the advancement element with respect to the housing, the instrument is configured to advance distal to the distal end of the housing, and a blood sample collection device integrated into the distal end of the housing.

Clause 7: The instrument advancement device of Clause 6, wherein an opening of the blood sample collection device is in fluid communication with a fluid path defined by the instrument.

Clause 8: The instrument advancement device of Clause 6 or Clause 7, wherein the blood sample collection device is configured for removal from the housing once a small volume blood sample has been received in the blood sample collection device for point-of-care testing, and wherein the small volume blood sample is 0.3 to 500 microliters or 0.3 to 100 microliters.

Clause 9: The instrument advancement device of any of Clauses 6-8, further comprising a fluid chamber access port defined in the distal end of the housing to receive the blood sample collection device.

Clause 10: The instrument advancement device of any of Clauses 7-9, wherein the fluid chamber access port includes a seal that prevents blood from exiting the housing after the blood sample collection device has been removed from the housing.

Clause 11: The instrument advancement device of any of Clauses 6-10, wherein the blood sample collection device is pre-attached to the fluid chamber access port.

Clause 12: The instrument advancement device of any of Clauses 6-11, wherein the fluid chamber access port includes a valve member that moves between an open position in which blood is free to flow into the blood sample collection device and a closed position in which the blood is prevented from entering the blood sample collection device.

Clause 13: An instrument advancement device, comprising a housing comprising a distal end and a proximal end; an instrument disposed within the housing; an advancement element, wherein in response to movement of the advancement element with respect to the housing, the instrument is configured to advance distal to the distal end of the housing, a blood sample access port defined in the housing, and an inner cylinder positioned within the housing that is rotatable relative to the housing.

Clause 14: The instrument advancement device of Clause 13, wherein the inner cylinder defines an aperture, in which the aperture is rotatable into alignment with the blood sample access port to permit blood to flow into a blood sample collection device operatively connected to the instrument advancement device and out of alignment with the blood sample access port to prevent the blood flowing into the blood sample collection device.

Clause 15: The instrument advancement device of Clause 13 or Clause 14, wherein the housing further comprises a handle operatively connected to the inner cylinder to rotate the inner cylinder relative to the housing.

Clause 16: The instrument advancement device of any of Clauses 13-15, wherein the inner cylinder defines an aperture, and wherein blood sample access port is rotatable into alignment with the aperture to permit blood to flow into a blood sample collection device operatively connected to the instrument advancement device and out of alignment with the aperture to prevent the blood flowing into the blood sample collection device.

Clause 17: An instrument advancement device, comprising a housing comprising a distal end and a proximal end; an instrument disposed within the housing; an advancement element, wherein in response to movement of the advancement element with respect to the housing, the instrument is configured to advance distal to the distal end of the housing, and a blood sample collection device operatively connected to the advancement element to collect a small volume blood sample of 0.3 to 500 microliters or 0.3 to 100 microliters.

Clause 18: The instrument advancement device of Clause 17, wherein the blood sample collection device is operatively connected to a proximal end of the advancement element.

Clause 19: The instrument advancement device of Clause 17 or Clause 18, wherein the advancement element defines a fluid path to fluidly connect the blood sample collection device to a fluid path defined by the instrument.

Clause 20: The instrument advancement device of any of Clauses 17-19, wherein the blood sample collection device is configured to be removed from the advancement element after the small volume blood sample has been received in the blood sample collection device for analysis by a point-of-care device.

### BRIEF DESCRIPTION OF THE DRAWINGS

Example embodiments will be described and explained with additional specificity and detail through the use of the accompanying drawings in which:

FIG. 1A is an upper perspective view of an example instrument advancement device, illustrating an example advancement element in an example initial or retracted position, according to some embodiments;

FIG. 1B is a longitudinal cross-sectional view of the instrument advancement device of FIG. 1A;

FIG. 1C is a longitudinal cross-sectional view of the instrument advancement device of FIG. 1A, illustrating the advancement element in the initial or retracted position according to some embodiments;

FIG. 1D is a cross-sectional view of the instrument advancement device of FIG. 1A along the line 1D-1D of FIG. 1A according to some embodiments;

FIG. 1E is an enlarged view of a portion of FIG. 1D according to some embodiments;

FIG. 1F is a cross-sectional view of the instrument advancement device of FIG. 1A along the line 1F-1F of FIG. 1A according to some embodiments;

FIG. 2 is a side view of an instrument advancement device according to some embodiments;

FIG. 3 is a side view of an instrument advancement device according to some embodiments;

FIG. 4 is a side view of an instrument advancement device according to some embodiments;

FIG. 5 is a side view of an instrument advancement device according to some embodiments with a sample access port closed;

FIG. 6 is a side view of an instrument advancement device according to some embodiments with a sample access port open; and

FIG. 7 is a side view of an instrument advancement device according to some embodiments.

### DESCRIPTION OF THE DISCLOSURE

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates and grammatical variants of those words or phrases.

The figures accompanying this application are representative in nature, and should not be construed as implying any particular scale or directionality, unless otherwise indicated. For purposes of the description hereinafter, the terms “upper”, “lower”, “right”, “left”, “vertical”, “horizontal”, “top”, “bottom”, “lateral”, “longitudinal” and derivatives thereof shall relate to the invention as it is oriented in the drawing figures. However, it is to be understood that the invention may assume various alternative variations and step sequences, except where expressly specified to the contrary. Hence, specific dimensions and other physical characteristics related to the embodiments disclosed herein are not to be considered as limiting.

The present disclosure is directed to a vascular access instrument delivery device that provides needle-free delivery of a probe or tube to a patient's vascular system for blood collection with integrated small volume blood sample collection devices for POC testing. These blood sample collection containers may be optimized to dispense blood to various POC systems and instruments, including cartridges for POC systems or test strips such as for glucose monitors. In one aspect or embodiment, the small volume blood sample discussed herein is 0.3 to 500 microliters. In a further aspect or embodiment, the small volume blood sample discussed herein is 0.3 to 100 microliters.

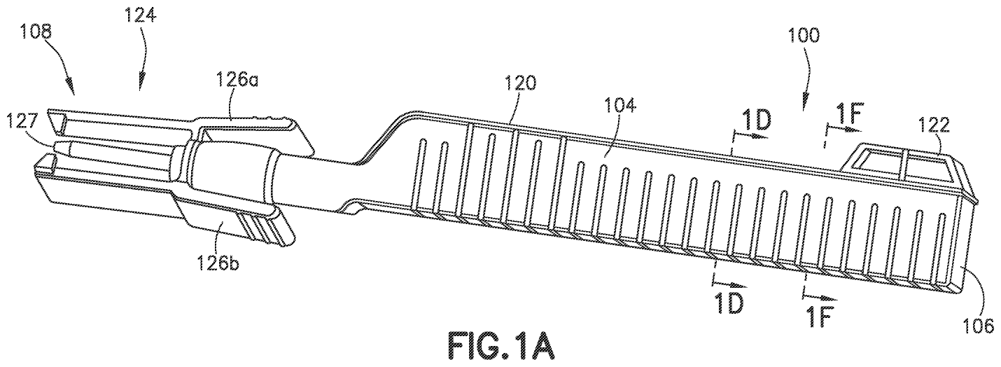

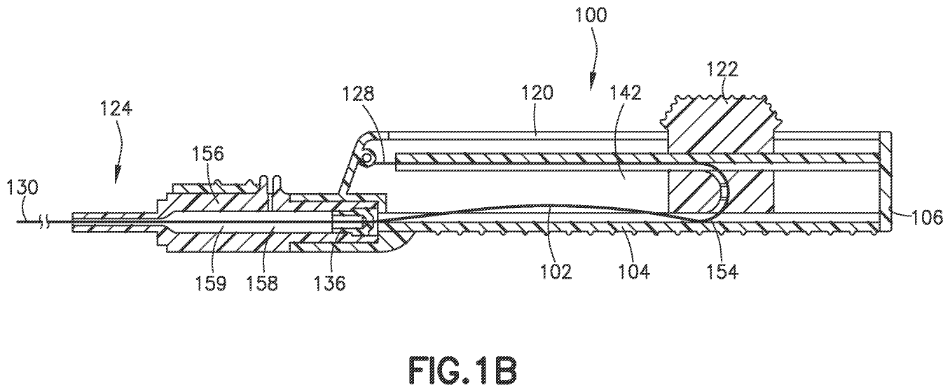

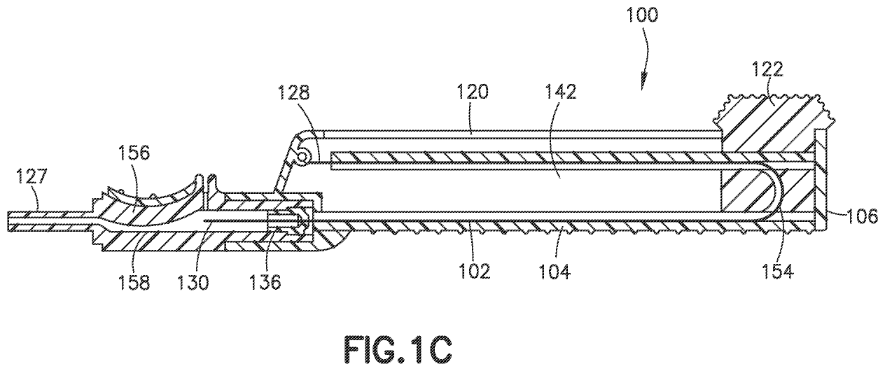

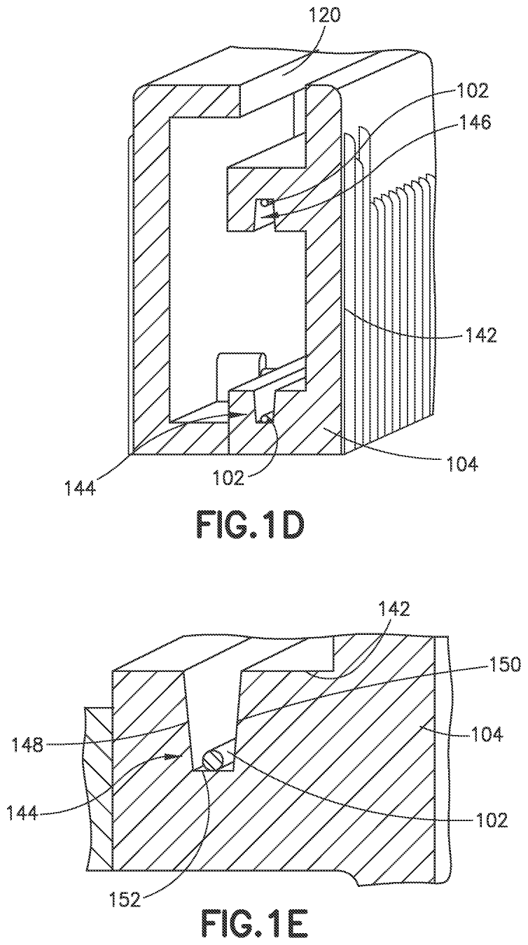

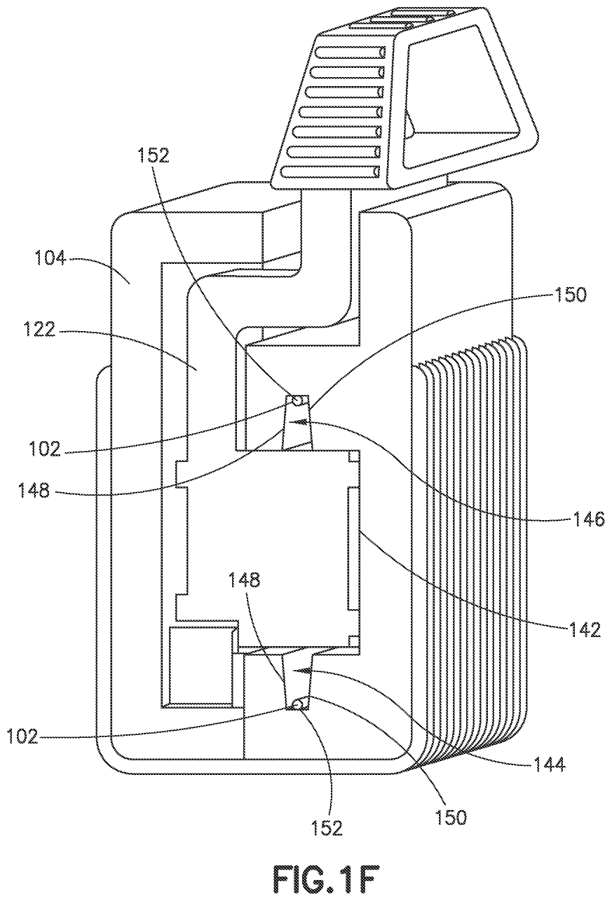

Referring now to FIGS. 1A-1F, in some embodiments, an instrument advancement device 100 may be configured to deliver an instrument 102 through a catheter of a catheter assembly. In some embodiments, the instrument 102 may be advanced through the catheter to push past any occlusions in the catheter or vasculature (e.g., thrombus or fibrin sheath at a tip of the catheter, vein collapse, valves, etc.) to create a clear pathway for fluid flow. In some embodiments, the instrument 102 may reduce or remove occlusions, improving patency of the catheter for medication and fluid delivery, as well as blood acquisition, during a dwell time of the catheter.

In some embodiments, the instrument 102 may include a guidewire, a probe, a guidewire or a probe with one or more sensors, or another suitable instrument. In some embodiments, the sensors may be used for patient or device monitoring and may include sensors measuring pressure, temperature, pH, blood chemistry, oxygen saturation, flow rate, or another physiological property.

In some embodiments, the catheter may include a peripheral IV catheter, a peripherally-inserted central catheter, or a midline catheter. In some embodiments, the catheter through which the instrument 102 may be delivered, may have been previously inserted into vasculature of a patient and may be dwelling within the vasculature when the instrument 102 is advanced through the catheter.

In some embodiments, the instrument 102 may be disposed within a housing 104, which may be configured to protect the instrument 102 from damage and/or contamination from a surrounding external environment. In some embodiments, the housing 104 may be rigid or semi-rigid. In some embodiments, the housing 104 may be made of one or more of stainless steel, aluminum, polycarbonate, metal, ceramic, plastic, and another suitable material. In some embodiments, the housing 104 may include a proximal end 106, a distal end 108, and a slot 120. In some embodiments, the slot 120 may extend parallel to a longitudinal axis of the housing 104.

In some embodiments, the instrument advancement device 100 may include an advancement element 122, which may extend through the slot 120 and may be configured to move linearly along the slot 120 between a retracted position illustrated, for example, in FIG. 1A, and an advanced position. In some embodiments, the user may pinch or grasp the advancement element 122 to move the advancement element 122 between the retracted position and the advanced position.

In some embodiments, the distal end 108 of the housing 104 may include a distal connector 124. In some embodiments, the distal connector 124 may include opposing lever arms 126a, 126b. In some embodiments, distal ends of the opposing lever arms 126a, 126b may be configured to move apart from each other in response to pressure applied to proximal ends of the opposing lever arms 126a, 126b. In some embodiments, in response to removal of the pressure applied to the proximal ends of the opposing lever arms 126a, 126b, the distal ends may move closer to each other and clasp a portion of the catheter assembly, such as a needleless connector, another connector, or a proximal end of a catheter adapter, for example. In some embodiments, the distal connector 124 may include a blunt cannula 127 or a male luer configured to insert into the portion of the catheter assembly.

In some embodiments, the distal connector 124 may include any suitable connector. For example, the distal connector 124 may include a threaded male luer, a slip male luer, a threaded male luer with a spin lock, a threaded male luer with a removable blunt cannula snap connection, a slip male luer with a removable blunt cannula snap connection, or another suitable connector. In some embodiments, the distal connector 124 may include one or more bond pockets, which may each be configured to receive an extension tube. In some embodiments, the distal connector 124 may be monolithically formed as a single unit with a body of the housing 104 that includes the slot 120.

In some embodiments, the instrument 102 may include a first end 128 and a second end 130. In some embodiments, movement of the advancement element 122 from the retracted position to the advanced position may cause the second end 130 of the instrument 102 to be advanced beyond the distal end 108 of the housing 104. In some embodiments, moving the advancement element 122 to the advanced position may introduce the instrument 102 into the catheter assembly and/or through the catheter. In some embodiments, in response to the instrument 102 being introduced into the catheter assembly and/or through the catheter, the instrument 102 may access a fluid pathway of the catheter assembly and/or the vasculature of a patient.

In some embodiments, the catheter of the catheter assembly with significant dwelling time within the vasculature may be susceptible to narrowing, collapse, kinking, blockage by debris (e.g., fibrin or platelet clots), and adhering of a tip of the catheter to the vasculature. Thus, blood withdrawal using the catheter may be difficult. In some embodiments, the instrument 102 may have a diameter less than a diameter of the catheter of the catheter assembly to provide access to the vasculature of the patient without any additional needle sticks. In some embodiments, the instrument 102 may clear the pathway for collecting a blood sample. Thus, in some embodiments, the instrument advancement device 100 may be used for needle-free blood collection and/or fluid infusion.

In some embodiments, an extension tube may be coupled to the instrument advancement device 100, and the extension tube may be used for blood collection and/or fluid infusion. In some embodiments, the extension tube may extend from a port of the housing 104. In some embodiments, a septum 136 may be within the housing 104 to enable the instrument 102 to advance and/or retract while maintaining a closed fluid path. In some embodiments, the instrument 102 may be configured to extend through the septum 136. In some embodiments, the septum 136 may be disposed proximal to a port and distal to the advancement element 122 in the advanced position. In some embodiments, the septum 136 may include silicone, rubber, an elastomer, or another suitable material. In some embodiments, the septum 136 may include an aperture, slit, or the like to accommodate the instrument 102 therethrough.

In some embodiments, an inner surface 142 of the housing 104 may include one or more grooves. For example, the inner surface 142 may include a first groove 144 and/or a second groove 146. In some embodiments, the first groove 144 and/or the second groove 146 may be disposed within the housing 104 between the proximal end 106 and the distal end 108. In some embodiments, the instrument 102 may be disposed within the first groove 144 and/or the second groove 146. In some embodiments, the first groove 144 and/or the second groove 146 may include a support wall 148, another support wall 150 opposite the support wall, and a bottom 152 extending between the support wall 148 and the other support wall 150. In some embodiments, the first groove 144 and/or the second groove 146 may be open opposite the bottom 152. In some embodiments, the first groove 144 and/or the second groove 146 may be linear and/or configured to guide the instrument 102 as the instrument 102 is advanced distally and/or retracted proximally.

In some embodiments, the advancement element 122 may include an arc-shaped channel 154, which may be U-shaped. In some embodiments, the instrument 102 may extend and move through the arc-shaped channel 154. In some embodiments, the first end 128 of the instrument 102 may be fixed. In some embodiments, the first end 128 of the instrument may be fixed within the housing 104. In some embodiments, in response to movement of the advancement element 122 a first distance, the second end of the instrument 102 may be configured to advance distally a second distance. In some embodiments, the second distance may be twice the first distance. In some embodiments, the second distance may be more than twice the first distance. In these and other embodiments, the instrument 102 may extend through multiple U-shapes or other arc-shapes. In some embodiments, the distal end 108 of the housing 104 may include a compressible portion 156 proximate a chamber 158 configured to hold blood. In some embodiments, the compressible portion 156 may be constructed of a flexible material, which may have a lower durometer than portions of the housing 104 surrounding the compressible portion 156. In some embodiments, a blood collection pathway 159 may extend within the chamber 158 and/or through the extension tube 132.

In some embodiments, because the first groove 144 and/or the second groove 146 are open opposite the bottom 152, the instrument 102 may tend to buckle in response to the advancement element 122 being advanced distally, as illustrated, for example, in FIG. 1B. Thus, in some embodiments, one or more support features may be used. The support features may be described further in U.S. Patent Application No. 63/164,976, filed Mar. 23, 2021, entitled VASCULAR ACCESS DEVICE TO REDUCE BUCKLING OF AN INSTRUMENT, which is herein incorporated by reference in its entirety.

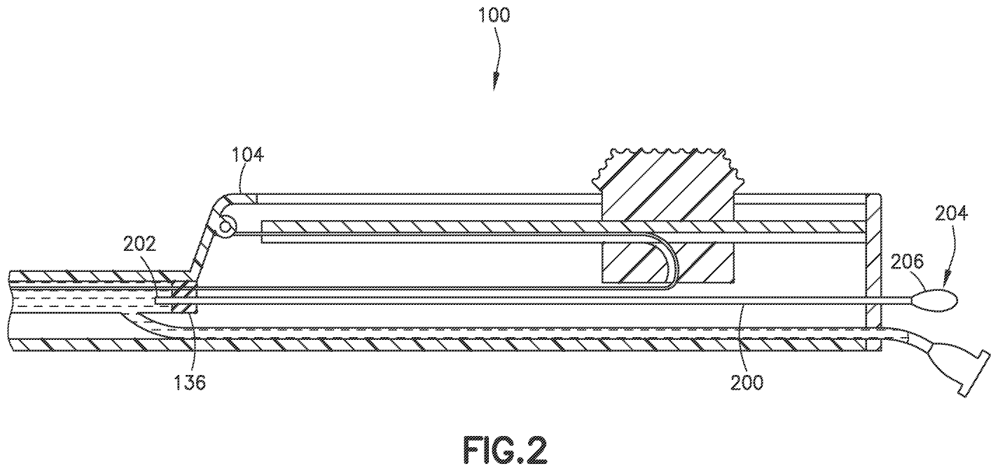

With reference to FIG. 2, according to one non-limiting embodiment or aspect of the present disclosure, the instrument advancement device 100 may include additional features for drawing blood from the blood path of the patient. In this embodiment, a blood sample collection tube 200 may be placed in the housing 104. In one example, the blood sample collection tube 200 may be a capillary tube. The blood sample collection tube 200 may be positioned so a tip 202 of the blood sample collection tube 200 is proximal to the septum 136. Once the instrument advancement device 100 has been used and blood is flowing through the fluid path, the blood sample collection tube 200 may be pushed through the septum 136 into the fluid path to collect a blood sample from the fluid path. After the blood sample has been collected, the blood sample collection tube 200 may be removed to dispense the blood sample into a point-of-care (POC) instrument or device for testing and analysis. In one embodiment, the blood sample collection tube 200 may be vented to allow air to escape from the inner cavity of the blood sample collection tube 200. A handle 204 of the blood sample collection tube 200 may include a flexible member 206, such as a bulb, that may be compressed or squeezed to create suction within the blood sample collection tube 200 to pull a blood sample into the blood sample collection tube 200 from the fluid path.

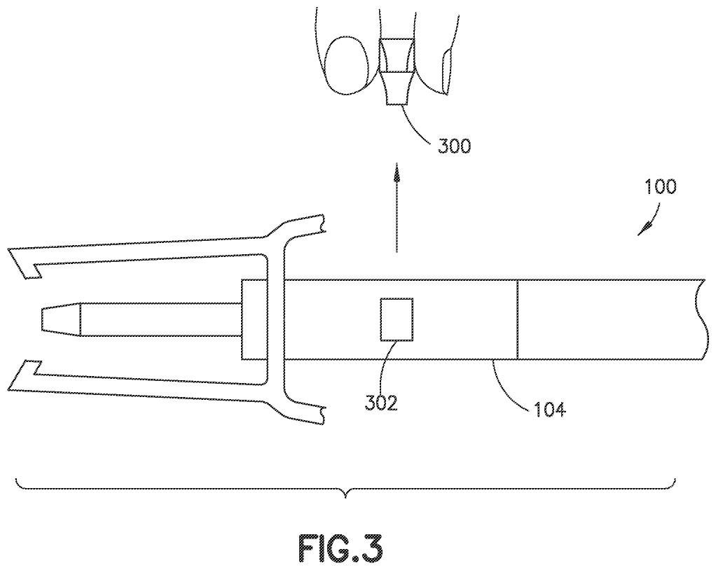

With reference to FIG. 3, according to one non-limiting embodiment or aspect of the disclosure, the instrument advancement device 100 may include additional features for drawing blood from the blood path of the patient. The instrument advancement device 100 may include a blood sample collection vial 300 integrated into the housing 104 of the instrument advancement device 100 near a distal end of the instrument advancement device 100. An opening 302 of the blood sample collection vial 300 may enter the fluid path of the instrument advancement device 100. After drawing blood through the instrument advancement device 100, a blood sample may be drawn up into the blood sample collection vial 300 via capillary action or by compressing a bulb on the blood sample collection vial 300 to create a vacuum. The blood sample collection vial 300 may then be removed from the instrument advancement device 100 to dispense the blood sample into a POC device or instrument for testing and analysis. The blood may be dispensed by compressing a bulb on the vial 300 to squeeze out the blood.

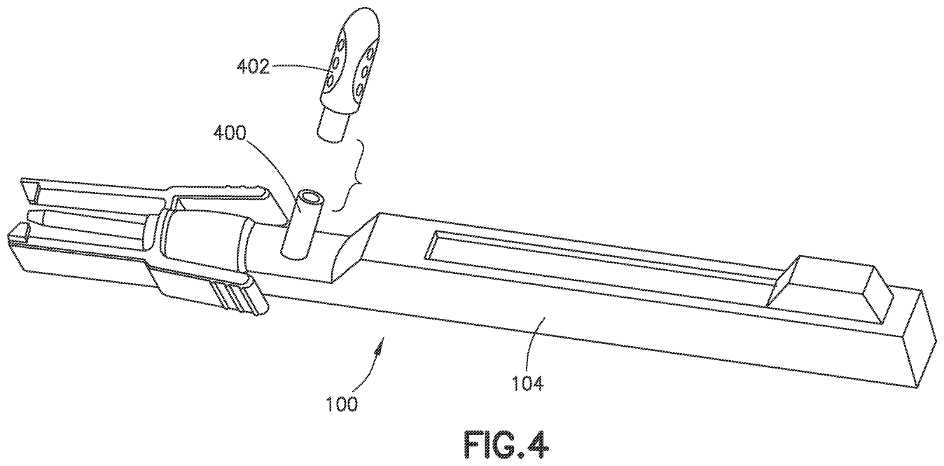

With reference to FIG. 4, according to one non-limiting embodiment or aspect of the disclosure, the instrument advancement device 100 may include additional features for drawing blood from the blood path of the patient. The instrument advancement device 100 may include a fluid chamber access port 400. A blood sample collection device 402 may be pre-attached to the fluid chamber access port 400. The blood sample collection device 402 may be removed after the user acquires a blood sample. A seal may be defined in the fluid chamber access port 400 to prevent blood from exiting the instrument advancement device 100 upon removal of the POC blood sample collection device 402.

A blood sample may enter the blood sample collection device 402 with an air vent via venous pressure, by a user applying a squeeze and fill step (user energized), or by a vacuum tube pressure collapsing an unvented, flexible acquisition device prior to filling when the vacuum tube is removed. It is also contemplated that other appropriate means for obtaining a blood sample using the blood sample collection device 402 may also be used with the present disclosure. In one example, the blood sample collection device 402 may initially not be connected to the fluid path to avoid pulling air into the instrument advancement device 100 or the blood sample collection device 402. After a large blood sample vacuum tubing collection is complete, the blood sample collection device 402 may be actuated to establish fluid communication with the instrument advancement device 100 and allow the blood sample collection device 402 to fill with a blood sample via venous pressure. In another example, the blood sample collection device 402 may include a valve feature that remains closed during the large blood sample vacuum tubing collection. After the large blood sample collection, the user may disengage or open the valve feature to establish fluid communication with the instrument advancement device 100. After a blood sample has been dispensed into the blood sample collection device 402, the valve feature may be closed to allow removal of the blood sample collection device 402 without blood leakage from the instrument advancement device 100.

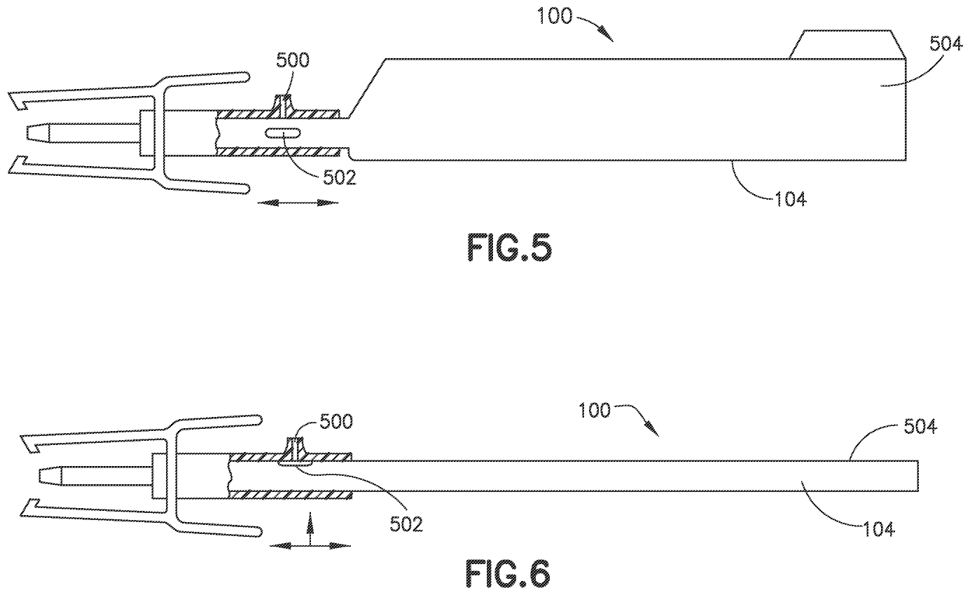

With reference to FIGS. 5 and 6, according to one non-limiting embodiment or aspect, the instrument advancement device 100 may include additional features for drawing blood from the blood path of the patient. The instrument advancement device 100 may include two concentric cylinders in the housing 104. The two concentric cylinders may be rotated relative to one another to open and close a sample access port 500 defined in the housing 104. An inner cylinder defines an aperture 502 that faces upwardly when in the closed position so that fluid only flows through a large sample fluid path through the housing 104. When the user is ready to collect the small blood sample, the sample access port fluid path is opened by rotating a handle 504 to align the aperture 502 of the inner cylinder to the sample access port 500 to establish fluid communication with the small sample fluid path. In another embodiment, the sample access port 500 may be configured so that the sample access port 500 rotates relative to the main fluid path of the instrument advancement device 100 to open and close the small sample fluid path.

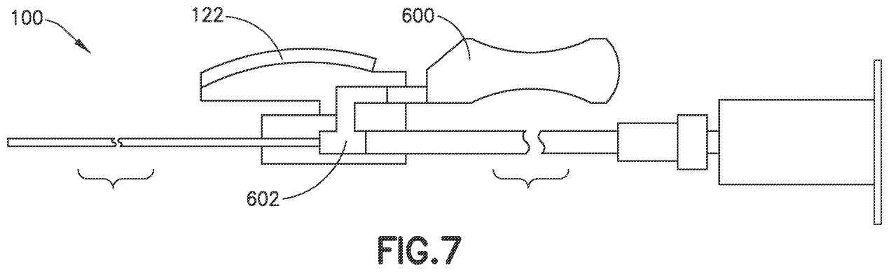

With reference to FIG. 7, according to one non-limiting embodiment or aspect, the instrument advancement device 100 may include additional features for drawing blood from the blood path of the patient. A blood sample collection device 600 may be positioned proximal to the advancement element 122. A fluid path 602 may be established between the large fluid path of the instrument advancement device 100 and the blood sample collection device 600 through the advancement element 122. After a blood sample is drawn through the instrument advancement device 100, the blood sample collection device 600 may be activated to collect a small volume blood sample through the fluid path 602. The blood sample collection device 600 may then be removed and used to dispense the blood sample to a POC instrument or device for testing and analysis.

In some of the embodiments of the instrument advancement device 100, such as those illustrated in FIGS. 3-6, the blood sample collection device may be vented to allow air to escape as venous pressure drives the blood sample into the blood sample collection device. The blood sample collection device may be compressible such that the blood sample may be dispensed when the blood sample collection device is compressed or squeezed.

The various examples of the instrument advancement device 100 described above provide the ability to a user to dispense a small volume of blood from the instrument advancement device 100 for POC testing and analysis. These various instrument advancement devices 100 also eliminate the need for separate collection and transfer devices for blood sample testing and analysis. These various instrument advancement devices 100 also eliminate the need for needle sticks and finger pricks commonly associated with POC blood collection.

While the present invention has been described in terms of the above detailed description, those of ordinary skill in the art will understand that alterations may be made within the spirit of the invention.

The invention claimed is:

1. An instrument advancement device, comprising:
a housing comprising a distal end and a proximal end;
an instrument disposed within the housing;
an advancement element, wherein in response to movement of the advancement element with respect to the housing, the instrument is configured to advance distal to the distal end of the housing, and
a blood sample collection tube insertable into the distal end of the housing, wherein the blood sample collection tube is configured to receive a small volume blood sample of 0.3 to 500 microliters.

2. The instrument advance device of claim 1, wherein the small volume blood sample is 0.3 to 100 microliters.

3. The instrument advancement device of claim 1, wherein the instrument includes a septum providing in a proximal end of the instrument, and wherein the blood sample collection tube is inserted into the instrument through the septum to receive the small volume blood sample.

4. The instrument advancement device of claim 1, wherein, after the small volume blood sample has been received in the blood sample collection tube, the blood sample collection tube is configured to be removed from the housing.

5. The instrument advancement device of claim 4, wherein the blood sample collection tube is configured to be operatively connected to a point-of-care system to test the small volume blood sample.

6. The instrument advancement device of claim 1, wherein the blood sample collection tube includes a flexible member on a proximal end of the blood sample collection tube to create suction to pull the small volume blood sample into the blood sample collection tube.

7. An instrument advancement device, comprising:
a housing comprising a distal end and a proximal end;
an instrument disposed within the housing;
an advancement element, wherein in response to movement of the advancement element with respect to the housing, the instrument is configured to advance distal to the distal end of the housing, and
a blood sample collection device integrated into the distal end of the housing.

8. The instrument advancement device of claim 7, wherein an opening of the blood sample collection device is in fluid communication with a fluid path defined by the instrument.

9. The instrument advancement device of claim 7, wherein the blood sample collection device is configured for removal from the housing once a small volume blood sample has been received in the blood sample collection device for point-of-care testing, and wherein the small volume blood sample is 0.3 to 500 microliters.

10. The instrument advancement device of claim 7, further comprising a fluid chamber access port defined in the distal end of the housing to receive the blood sample collection device.

11. The instrument advancement device of claim 8, wherein the fluid chamber access port includes a seal that prevents blood from exiting the housing after the blood sample collection device has been removed from the housing.

12. The instrument advancement device of claim 7, wherein the blood sample collection device is pre-attached to the fluid chamber access port.

13. The instrument advancement device of claim 7, wherein the fluid chamber access port includes a valve member that moves between an open position in which blood is free to flow into the blood sample collection device and a closed position in which the blood is prevented from entering the blood sample collection device.

14. An instrument advancement device, comprising:
a housing comprising a distal end and a proximal end;
an instrument disposed within the housing;
an advancement element, wherein in response to movement of the advancement element with respect to the housing, the instrument is configured to advance distal to the distal end of the housing,
a blood sample access port defined in the housing, and
an inner cylinder positioned within the housing that is rotatable relative to the housing.

15. The instrument advancement device of claim 14, wherein the inner cylinder defines an aperture, in which the aperture is rotatable into alignment with the blood sample access port to permit blood to flow into a blood sample collection device operatively connected to the instrument advancement device and out of alignment with the blood sample access port to prevent the blood flowing into the blood sample collection device.

16. The instrument advancement device of claim 14, wherein the housing further comprises a handle operatively connected to the inner cylinder to rotate the inner cylinder relative to the housing.

17. The instrument advancement device of claim 14, wherein the inner cylinder defines an aperture, and wherein the blood sample access port is rotatable into alignment with the aperture to permit blood to flow into the blood sample collection device operatively connected to the instrument advancement device and out of alignment with the aperture to prevent the blood flowing into the blood sample collection device.

18. An instrument advancement device, comprising:
a housing comprising a distal end and a proximal end;
an instrument disposed within the housing;
an advancement element, wherein in response to movement of the advancement element with respect to the housing, the instrument is configured to advance distal to the distal end of the housing, and
a blood sample collection device operatively connected to the advancement element to collect a small volume blood sample of 0.3 to 500 microliters.

19. The instrument advancement device of claim 18, wherein the blood sample collection device is operatively connected to a proximal end of the advancement element.

20. The instrument advancement device of claim 18, wherein the advancement element defines a fluid path to fluidly connect the blood sample collection device to a fluid path defined by the instrument.

21. The instrument advancement device of claim 18, wherein the blood sample collection device is configured to be removed from the advancement element after the small volume blood sample has been received in the blood sample collection device for analysis by a point-of-care device.

22. The instrument advancement device of claim 18, wherein the small volume blood sample is 0.3 to 100 microliters.
